(19) United States
(12) Patent Application Publication
(10) Pub. No.: US 2004/0115249 A1
(43) Pub. Date: Jun. 17, 2004

(54) Ionic compounds of quaternary ammonium cations with anions of preservation acids, preparation thereof, and use thereof for preservation

(76) Inventors: Nico N. Raczek, Kelkheim (DE); Christoph Mollenkopf, Frankfurt (DE)

Correspondence Address: PROPAT, L.L.C., 2912 CROSBY ROAD, CHARLOTTE, NC, 28211-2815, US

(21) Appl. No.: 10/730,180

(22) Filed: Dec. 8, 2003

(30) Foreign Application Priority Data
Dec. 12, 2002 (DE) 102 58 003.0

(51) Int. Cl. A61K 7/075; A61K 7/08; A23K 1/165; A23K 1/17

(52) U.S. Cl. 424/442; 424/70.28

(57) ABSTRACT

The present invention relates to ionic compounds of quaternary ammonium cations with anions of preservative acids, which ionic compounds can be used for preserving, for example, cosmetics, laundry detergents and cleaning compositions, pharmaceuticals, requisites intended for contact with humans or foods, or as preservative and disinfectant agents in industrial products, or in foods, feedstuffs etc. In addition, the invention relates to formulations which comprise the inventive compounds and optionally a stabilizer and/or a formulation aid.

### FIELD OF THE INVENTION

The present invention relates to ionic compounds of quaternary ammonium cations with anions of preservative acids, which ionic compounds can be used for preserving, for example, cosmetics, laundry detergents and cleaning compositions, pharmaceuticals, requisites intended for contact with humans or foods, or as preservative and disinfectant agents in industrial products, or in foods, feedstuffs etc. In addition, the invention relates to formulations which comprise the inventive compounds and optionally a stabilizer and/or a formulation aid.

### BACKGROUND OF THE INVENTION

Worldwide, there is a trend to decrease the use of additives in foods, cosmetics, pharmaceuticals, feedstuffs etc., or to replace them completely by novel compounds. In particular in the case of preservative compositions, for example, the use of compounds releasing halogen (for example dichlorophene), antibiotics, heavy-metal-containing compounds, sulfur-containing substances (for example sulfur dioxide), substances releasing formaldehyde (for example hexamethylenetetramine), boric acid derivatives (for example sodium tetraborate) and many others is increasingly being restricted. This applies in particular to the substances which are used for nutrition or come into direct contact with the human body, for example cosmetics and pharmaceuticals, requisites intended for contact with humans or foods, laundry detergents and cleaning compositions, foods, feedstuffs, pet foods and wastes of the food industry. Other preservatives come under the class of antibiotics (for example natamycin), or their use will be further restricted in future by legal provisions (such as trichloroacetic acid, sulfur dioxide and carbon disulfide, nitrates, nitrites, esters of p-hydroxybenzoic acid, ethylene oxide etc.).

In the case of the abovementioned preservatives, in particular the frequently only highly specific activity toward certain microorganisms or groups of microorganisms has a highly disadvantageous effect on their use. Thus, for example nitrites and nitrates have good activity toward certain bacteria; in contrast, activity toward molds or yeasts is not observed. Biphenyl has good activity toward some yeasts and molds, but has no activity toward bacteria. The activity of the above-mentioned preservative acids (sorbic acid, propionic acid or benzoic acid) toward bacteria is generally very low. Thus, for example, sorbic acid has such a selective action, that sorbic-acid-containing nutrient media are used in microbiological diagnostics for growing clostridia, lactic acid bacteria and coagulase-positive staphylococci.

It is known that quaternary ammonium salts (quats) have antimicrobial action. The disinfectant activity of the quats, however, is limited to their bactericidal action at elevated pHs. Molds and yeasts are inhibited only to a very limited extent at elevated pHs. The pHs of foods are virtually exclusively in the acidic range. Many pharmaceuticals, acid cleansers or cosmetics are made acidic for reasons of activity. Quats, therefore, cannot be used for preservation in these ranges, in particular if action against yeasts and molds is required.

Differing product properties, in particular low pHs, of the products to be preserved, lead to the fact that a generally optimum preservative cannot be specified, but product-specific solutions are always required in preservation. However, in many cases, such solutions mean that, in addition to a preservative, other substances must be added and there is thus the necessity of adding and keeping available various preservatives, for example, or synergistically acting substances simultaneously in high amounts. Furthermore, even at legally authorized concentrations of preservatives (e.g. approximately 3 750 ppm of calcium propionate in sliced bread in the EU, glutaraldehyde at 1 000 ppm in cosmetics in the EU) excessive adverse sensory changes occur, which are frequently rejected by consumers.

### BRIEF SUMMARY OF THE INVENTION

It is an object underlying the invention, therefore, to provide defined substances which not only have a bacteria-destroying or bacteria-reducing microbiostatic action, but at the same time have an activity toward molds and yeasts at relatively low pH ranges and which can readily be employed in the foods, cosmetics or feed industry or related industrial branches.

Low pH ranges are to be taken to mean a pH of <7.0, in particular 2.5-6.0.

### DETAILED DESCRIPTION OF THE INVENTION

The object mentioned above is achieved by substances whose parent bodies are quaternary ammonium cations which, instead of the customary chloride or bromide anions, have anions of a preservative acid such as sorbates or benzoates. The invention accordingly relates to compounds of the formula:

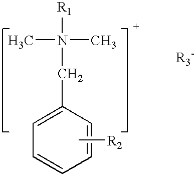

where

R₁=C₈-C₂₀-alkyl

R₂=H, methyl, ethyl, propyl and isopropyl

R₃=sorboyl (trans, trans CH₃—CH═CH—CH═CH—COO), benzoyl

One potential way of preparing such compounds is exchanging the halide anions of a quaternary ammonium halide for sorbate or benzoate anions, starting from the commercially conventional quaternary ammonium salts such as n-alkyldimethylbenzylammonium chloride. For this, for example, the quaternary ammonium salts are dissolved in water and an aqueous solution of a sorbic acid salt or benzoic acid salt such as potassium sorbate or sodium benzoate is added. After the water has been removed, the remaining reaction product is taken up in an organic solvent such as ethanol or acetone and in this way separated from the inorganic salt of the corresponding counterions, for example potassium chloride. The inventive quaternary ammonium salts are obtained as white waxy substances. Alternatively, the quaternary ammonium salt can be reacted with a base, for example sodium hydroxide. Then, sorbic acid or benzoic acid is added in equimolar amounts to the reaction mixture. The reaction mixture is worked up further as described in the above preparation process.

Surprisingly, the inventive compounds exhibit a markedly improved antimicrobial action against numerous different microorganisms.

Excellent action not only at elevated pHs, but also at low pHs (<7) was additionally surprising.

The addition of synergists or further preservatives or agents influencing pH was not necessary for this.

The inventive compounds can be used either alone as such, but in particular as solutions, preferably as aqueous or alcoholic solutions. If necessary, it is also possible to prepare formulations in which the inventive compounds are admixed with customary additives such as suspension aids, thickeners, carriers, film-formers or the like. To form coatings on foods and for coating packaging media, expediently carriers or coating- or film-formers are used. Suitable substances for this are, in particular, starch, starch ethers, oxidized or degraded starch, cellulose ethers, alginates, gelatin and polyvinyl alcohol.

The inventive compounds may be used in all sectors where conventional preservatives are used. Below, a description is given of some products in which the inventive compounds can preferably be used, together with the respective expedient concentrations (in each case based on the mass of the product, unless stated otherwise):

I. Cosmetics

Cosmetics are to be taken to mean compositions which are used externally on the human body for cleaning and care or for affecting the appearance or aroma of the body. These include, for example:

I.a Shaving Creams, After-Shave Care Products

The inventive compounds can be used for preserving creams and lotions having low alcohol contents, using about 0.01-0.2% by weight.

I.b UV Protection Products

For example in sun protection creams or lotions, 0.02-0.3% by weight of the inventive compounds should be used for adequate preservation.

I.c Hair Coloring Products

An expedient concentration for use in products for coloring hairs, eyelashes and eyebrows is about 0.1% by weight, preferably 0.01-0.2% by weight.

I.d Moist Cloths

To protect against recontamination and for prolonged storage life, inventive compounds can be used, for example in moist baby-cleaning cloths, at concentrations between 0.02 and 0.35% by weight.

I.e Hand Disinfectants

As additive to disinfectants for hands, inventive substances can be used alone or in a mixture with other disinfectant substances at concentrations between 0.02 and 0.5% by weight.

II. Requisites Intended for Contact with Humans or Foods

Compositions which are used for cleaning articles which can come into contact with foods or act on these.

II.a Post-Rinse Laundry Aids

The inventive compounds can be used to preserve laundry in post-rinse laundry aids, such as fabric conditioners, in order to obtain a disinfectant action on the laundry thus treated. Suitable concentrations in use are between 0.005 and 0.5% by weight, preferably 0.01-0.35% by weight.

II.b Dishwashing Compositions

Mixtures of various surfactants, a possible addition of stabilizers, fragrance oils, colorings etc. for commercial cleaning of bottles, barrels, glasses etc. or for use in conventional domestic machines or for dishwashing by hand can comprise 0.05-0.3% by weight of inventive substances.

II.c Cleaning Compositions

Surface-cleaning compositions, in particular those which are used for decreasing the microbial count on surfaces in the food industry, cosmetics industry, pharmaceuticals industry or food- or human-contact requisites industry, are permanently preserved with 0.05-0.3% by weight of inventive substances.

III Foods

III.a Fruit, Fruit Products, Dried Fruit, Jams and Marmalades

An expedient concentration in use is about 0.25% by weight, preferably 0.05 to 0.35% by weight. For surface treatment, alcoholic or aqueous solutions can be sprayed on, or the fruit is dipped into suitable solutions containing active compound at up to 10% by weight. In the case of treated and processed products, the inventive products should expediently be added toward the end of the heating process; this applies in particular to heated products.

III.b Vegetables, Pickled Vegetables, Delicatessen Salads and Sausage Sauces

An expedient concentration in use is about 0.25% by weight, preferably 0.05 to 0.4% by weight. For surface treatment alcoholic or aqueous solutions can be sprayed on or the fresh vegetables can be dipped into suitable solutions containing active compounds at up to 10% by weight. In the case of treated and processed products such as preserved pickled vegetables such as pickled gherkins, mixed pickles and the like and lactic-fermented vegetables such as sauerkraut and olives, the inventive compounds are customarily added to the make-up liquids in amounts of 0.005-0.4% by weight, preferably 0.05 to 0.3% by wight. In the same concentration range, they are also suitable for use in delicatessen salads, sausage sauces and related products, such as mustard.

III.c Baked Goods and Doughs

The inventive compounds may be used without problems in baked goods of the most varied types which are susceptible to decay, baked goods fillings, partially baked and prebaked products and ready-mixed doughs. Expedient quantities for this, are, depending on product and the desired shelf life extension, up to 0.75% by weight, preferably 0.05 to 0.5% by weight.

III.d Cheese

An addition of 0.005-0.5% by weight, preferably 0.05 to 0.35% by weight, based on the total cheese loaf, is generally sufficient for preserving ripened cheese. Addition to the brine bath maintains the cheese thus treated mold-free for several weeks. Preferably, 1-10 g/l of the compounds, depending on solubility, are added to the brine baths of hard cheeses. A surface treatment with aqueous solution by dipping or spraying with aqueous solutions or suspensions of the inventive compounds at a concentration of 10 g/l and after-treatment periods of 1 to 5 weeks keeps ripening cheese free from mold. In the case of cheeses which do not permit spray treatment, it is also possible to apply a suspension which has been made viscous. In the case of processed cheese, the inventive compounds can be added together with the emulsifying salts. Likewise, in the case of soft farmer's cheese and fermented milk products, addition of 0.005-0.5% by weight, preferably 0.05 to 0.25% by weight of the inventive compounds produces good extension of keeping quality.

III.e Fat Emulsions

In fat emulsions such as margarine, mayonnaise, salad sauces and dressings, up to 0.75% by weight, preferably 0.05 to 0.5% by weight, can be used. The addition is expediently made to the aqueous phase before emulsifying.

III.f Meat Products and Fish Products

Meat products and fish products, for example pates and fish marinades, may be preserved in most cases with 0.005-0.5% by weight, preferably 0.05 to 0.25% by weight, of the inventive compounds. In the case of particularly perishable products such as cooked crustacea, however, up to 1.0% by weight may be required.

III.g Surface Treatment and Coatings

The surface treatment of foods, for example meat products such as matured sausages or ham and dried meat can be carried out in the same manner and at the same quantities as with ripened cheeses.

IV. Packaging

By introducing the inventive compounds into coatings of foods or industrial products such as packaging materials, a bacteriostatic effect is achieved. Using about 1 to 10 g of the inventive compounds/m², in the case of packaging films or packaging paper a protective action is achieved against the formation of microorganisms, in particular mold formation, underneath the packaging, when this is in direct contact with the packaged goods. The coated packaging can be used not only to retain freshness of fresh food such as meat, fish, cheese and cheese products, but also for packaged foods of the most varied types such as baked goods, pasta and “convenience food” in the broadest sense.

V. Animal Feed and Products Suitable as Feedstuffs

Addition of the inventive compounds likewise extends the keeping quality of animal feed. These also include products which are suitable for use as animal feed, for example silages, brewer's spent grains, pomace, brewer's yeast, distiller's spent wash and various food wastes. The inventive compounds can be added in suitable powder form dried to the feed, before further processing (for example extrusion) or, in solution, can be sprayed on or added in dissolved form in a mixture. For these purposes, concentrations up to 2.5% by weight, preferably 0.05 to 1.5% by weight, are employed.

VI. Drugs

To preserve water-containing pharmaceuticals, for extending keeping quality, the inventive compounds are added to these in a concentration of 0.005-0.5% by weight, preferably 0.05 to 0.25% by weight.

VII. Industrial Products

To preserve requisites intended for contact with humans or foods, including those which are intended for cleaning and care of machines and apparatuses in the food industry, as for microbially susceptible industrial products, the inventive compounds can be added in concentrations of 0.05-0.5% by weight, preferably 0.1 to 0.4% by weight.

VIII. Wood

To preserve wood, wooden pallets, sawn timber and timber fragments, the inventive compounds can be added at concentrations of 0.05 to 2.0% by weight.

IX. Paints

To preserve emulsion paints, the inventive compounds can be added in concentrations of 0.05 to 2.0% by weight.

Further examples for the use of the inventive compounds are products which come into contact with children, such as finger paints, kneadable compounds, coatings on wooden toys etc.

The invention will be described by the examples below:

### EXAMPLE 1

Preparation of n-alkyldimethylbenzylammonium Sorbate

40 g of n-alkyldimethylbenzylammonium chloride (approximately 0.1 mol, n-alkyl 60% C₁₄, 30% C₁₆, 5% each C₁₂ and C₁₈) were dissolved in water and charged. 15 g of potassium sorbate in 20 g of water were added thereto. At 50° C. and 30 mbar, the water was removed from the reaction mixture by distillation. The waxy residue was taken up in 50 ml of ethanol. The potassium chloride precipitates as a deposit and is filtered off. The ethanol was removed from the filtrate by distillation. This process was repeated twice further. After the final removal of the ethanol, the product is obtained as white residue.

### EXAMPLE 2

Preparation of n-alkyldimethylethylbenzylammonium Sorbate

40 g of n-alkyldimethylethylbenzylammonium chloride (approximately 0.1 mol, n-alkyl 68% C₁₂, 32% C₁₄) were dissolved in water and charged. 15 g of potassium sorbate in 20 g of water were added thereto. At 50° C. and 30 mbar, the water was removed from the reaction mixture by distillation. The waxy residue was taken up in 50 ml of ethanol. The potassium chloride precipitates as deposit and is filtered off. The ethanol was removed from the filtrate by distillation. This process was repeated twice further. After the final removal of the ethanol the product is obtained as white residue.

### EXAMPLE 3

Preparation of n-alkyldimethylbenzylammonium Benzoate

40 g of n-alkyldimethylbenzylammonium chloride (approximately 0.1 mol, n-alkyl 60% C₁₄, 30% C₁₆, 5% each C₁₂ and C₁₈) were dissolved in water and charged. 14.4 g of sodium benzoate in 20 g of water were added thereto. At 50° C. and 30 mbar, the water was removed from the reaction mixture by distillation. The waxy residue was taken up in 50 ml of ethanol. The potassium chloride precipitates as deposit and is filtered off. The ethanol was removed from the filtrate by distillation. This process was repeated twice further. After the final removal of the ethanol the product is obtained as white residue.

### EXAMPLE 4

Preparation of n-alkyldimethylethylbenzylammonium Benzoate

40 g of n-alkyldimethylethylbenzylammonium chloride (approximately 0.1 mol, n-alkyl 68% C₁₂, 32% C₁₄) were dissolved in water and charged. 14.4 g of sodium benzoate in 20 g of water were added thereto. At 50° C. and 30 mbar the water was removed from the reaction mixture by distillation. The waxy residue was taken up in 50 ml of ethanol. The potassium chloride precipitates as deposit and is filtered off. The ethanol was removed from the filtrate by distillation. This process was repeated twice further. After the final removal of the ethanol, the product is obtained as white residue.

### EXAMPLE 5

The product obtained in example 2 was used in a test of antimicrobial activity. Very low minimum inhibitory concentrations were found for microorganisms from different sectors (molds, yeasts, bacteria).

1. A substance comprising a compound of the formula 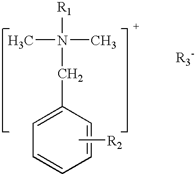
where
R₁=C₈-C₂₀-alkyl
R₂=H, methyl, ethyl, propyl and isopropyl
R₃=sorboyl (trans, trans CH₃—CH═CH═CH═CH—COO), benzoyl.

2. A food having a content of a compound as claimed in claim 1.

3. An animal feed having a content of a compound as claimed in claim 1.

4. A cosmetic having a content of a compound as claimed in claim 1.

5. An industrial product having a content of a compound as claimed in claim 1.

6. A requisite intended for contact with humans or foods which has a content of a compound as claimed in claim 1.

7. A packaging having a content of a compound as claimed in claim 1.

8. A drug having a content of a compound as claimed in claim 1.

9. A preservative comprising a compound as claimed in claim 1.

10. A surface treatment comprising a compound as claimed in claim 1.

11. A surface treatment as claimed in claim 10 for fruit or vegetables.

12. Food or hospital products comprising the surface treatment as claimed in claim 10.

13. A composition comprising a compound as claimed in claim 1 and an additive.

14. The composition as claimed in claim 13, wherein the additive is one or more substances selected from the group consisting of suspension aids, thickeners, carriers and film-formers.

15. A process for preparing the compound as claimed in claim 1, which comprises exchanging the halide anion of a quaternary ammonium halide for a sorbate or benzoate anion.
